(19) United States
(12) Patent Application Publication
(10) Pub. No.: US 2019/0298172 A1
(43) Pub. Date: Oct. 3, 2019

(54) Wearable Biosensor Ring

(71) Applicant: University of Massachusetts, Boston, MA (US)

(72) Inventors: MD Shaad Mahmud, New Bedford, MA (US); Honggang Wang, Shrewsbury, MA (US)

(21) Appl. No.: 16/294,636

(22) Filed: Mar. 6, 2019

Related U.S. Application Data
(60) Provisional application No. 62/639,571, filed on Mar. 7, 2018.

(51) Int. Cl. A61B 5/00; A61B 5/0205; A61B 5/16; A61B 5/11

(52) U.S. Cl. CPC A61B 5/0022; A61B 5/0205; A61B 5/165; A61B 5/6829; A61B 5/01; A61B 5/1117; A61B 5/747; A61B 5/1118; A61B 5/6826

(57) ABSTRACT

A ring includes a body. The ring also includes a microprocessor, located within the body. The ring also includes an EDA sensor in communication with the microprocessor and located within the body.

### FEDERALLY SPONSORED RESEARCH OR DEVELOPMENT

This invention was made with Government support under Grant Nos. RO1DA033323-01A1, 1UL1RR031982-01 awarded by the National Institutes of Health to Dr. Hua Fang, University of Massachusetts Dartmouth and Grant Nos. CCSS1407882, IIS1401711 and 1429120 award by the National Science Foundation. The Government has certain rights in the invention.

### BACKGROUND

Advancements in miniaturized electronics and smart sensors combined with a broad platform of smart phones, big data, cloud service and wireless communication have not only empowered wearable technology, they have also increased users life expectancy. This is done through a range of applications including; tracking physical activity, personalized health care, and recommendations for enhancing user experience.

### SUMMARY

In general, one innovative aspect of the subject matter described in this specification can be embodied in a ring that includes a body. The ring also includes a microprocessor, located within the body. The ring also includes an EDA sensor in communication with the microprocessor and located within the body.

In general, another innovative aspect of the subject matter described in this specification can be embodied in a ring that includes a body. The ring also includes a microprocessor, located within the body. The ring also includes a pulse oximeter in communication with the microprocessor and located within the body.

Other embodiments of this aspect include corresponding computer systems, apparatus, and computer programs recorded on one or more computer storage devices, each configured to perform actions and methods as described herein. A system of one or more computers can be configured to perform particular actions by virtue of having software, firmware, hardware, or a combination of them installed on the system that in operation causes or cause the system to perform the actions. One or more computer programs can be configured to perform particular actions by virtue of including instructions that, when executed by data processing apparatus, cause the apparatus to perform the actions.

In some implementations, the ring may optionally include one or more of the following features, alone or in combination. The body may include a pulse oximeter in communication with the microprocessor. The thickness of the body may be less than 2 mm. The ring may include a button and the microprocessor is configured to send a signal to a computing system in response to the button being pressed. The signal may be used to alert a third party. The ring may include an accelerometer. The microprocessor may be configured to identify signals of the accelerometer consistent with the wearer falling. The ring may be in communication with a computer system, the computer system configured to maintain health history information provided by the ring. The microprocessor may be configured to monitor the physiological condition of the wearer.

### BRIEF DESCRIPTION OF THE DRAWINGS

FIG. 1 is a block diagram of the proposed sensor architecture for Wearable biosensor ring.

FIG. 2 illustrates a Wearable biosensor ring (a) left-top and right-top point of view and (b) device with cover and belt.

FIG. 3 is a graph of peaks obtained from maximum of PPG for a wearer of the Wearable biosensor ring.

FIG. 4A-D are a time series data of EDA, Heart Rate, motor activity and Skin temperature.

Like reference numbers and designations in the various drawings indicate like elements.

### DETAILED DESCRIPTION

Described herein is a new type of wearable biosensor ring. Generally wearable sensors can be defined as; a combination of systems that can be worn anywhere on the human body to continuously and remotely observe an individual's activity without hindering the natural movement of the wearer. The wearable biosensor ring, in addition to providing continuous monitoring of a wearers physiological information. Moreover, the wearable biosensor ring can be used with machine learning systems to provide an indicator of early disease detection and prediction. The wearable biosensors ring uses a wireless unit to transmit the information enclosed in the wearable biosensor ring. Data from the device can be stored in a database so the data may be analyzed/processed to detect and predict possible critical situations in a wearer's health.

Recently, several commercial devices have started to emerge in the sports, fitness, and tele-medicine markets that are non-invasive and easy to use. Wrist-worn devices are one of the most popular wearables available in the market and can be found in both wristwatch and wrist band forms. Most wrist-worn devices work as an activity/fitness tracker, however; they lack the ability to measure electrodermal activity (EDA). EDA or galvanic skin resistance (GSR) measures changes in resistance at the surface of human skin. EDA (and GSR) reflects changes in the sympathetic nervous system (SNS). EDA is a prime biomarker for the detection of different emotional states due to the automatic responses of skin during changes in sweating, piloerection, and vasomotor levels. So, in addition to heart rate and activity monitoring, which is the key parameter for wearable sensors, having information about EDA and skin temperature can also give detailed information about a wearer's personal health. However, most the above-mentioned systems take the form of a wrist worn, chest wrap, or smart garment device.

Although there are a multitude of challenges when it comes to designing a ring biosensor, there are at least three major challenges when it comes to measuring SNS activity with this type of device. A first challenge is placing all of the electrical components together so that they can fit in a volume the size of a ring, while still keeping in mind the necessity for the ring to be low-weight and be comfortable for the user. A second challenge is creating a proper balance in between low computational complexity for real-time data processing and signal denoising for reducing motion artifacts. A third challenge is having the capability to transmit the acquired signal with a minimum quantity of power while also keeping a high accuracy level. As for the reliability of measuring EDA and heart rate variability by wearables, there are very few articles and review papers that are available to date on the subject. There are even less papers that discuss ring biosensors because, to the best knowledge of the authors of those corresponding papers, there is no currently available system that can measure EDA and skin temperature levels that also has the ability to fit into the volume of a compact ring-worn sensor.

The wearable biosensor ring can accurately measure EDA, HR, locomotion and temperature in a fully integrated system. The wearable biosensor ring is designed to measure EDA, HR, locomotion and temperature; provide real-time data transmission, as a result, keeping low computational complexity; providing an ultra-low power consumption using hibernation mode; and provide a cost-effective solution. The wearable biosensor ring was tested on several subjects in a laboratory setting for three days. The model was also tested for longer periods of time in the field.

The wearable biosensor ring (Wearable biosensor ring) can provide a wearable biofeedback system, that has the goal of enhanced accuracy and user-friendliness typical of wearable commercial products. The wearable biosensor ring can obtain multiparameters of vital signs, including custom made hardware architecture, signal pre-processing, power management and seamless connectivity. It can also have an application platform and PC based graphical user interface (GUI) software to allow quick and robust integration of several signal processing algorithms. The architecture of the Wearable biosensor ring can be capable of managing a network of sensors where multiple sensor nodes (rings) can connect to a single host (for example, in some implementations, eight rings can connect to a single host). Cross-talk in between sensor and node can also occur. The Wearable biosensor ring can include three major components: 1) Hardware design, where the sensors, which collect physiological data and communicate with the hardware; 2) the wireless network protocol to communicate in between hardware and software; and 3) Software design that includes the processing unit to extract and analyze the data to a meaningful level.

A key purpose of a wearable biosensor is to collect vital information and help users in different physiological conditions by providing necessary the tools without any kind of intervention. The proposed system can deliver all the requirements of a wearable sensor and to develop of the prototype, several design requirements were considered. Mainly 1) Non-invasiveness and easy to use, which requires a light and miniaturized size; 2) Design of a robust analog front end (AFE) for low latency in data processing; 3) a platform for data storage (Cloud and Local), 4) a strong signal processing algorithm for high quality and real-time data acquisition; 5) a cost effective and energy efficient system; 6) scalability for ubiquitous use.

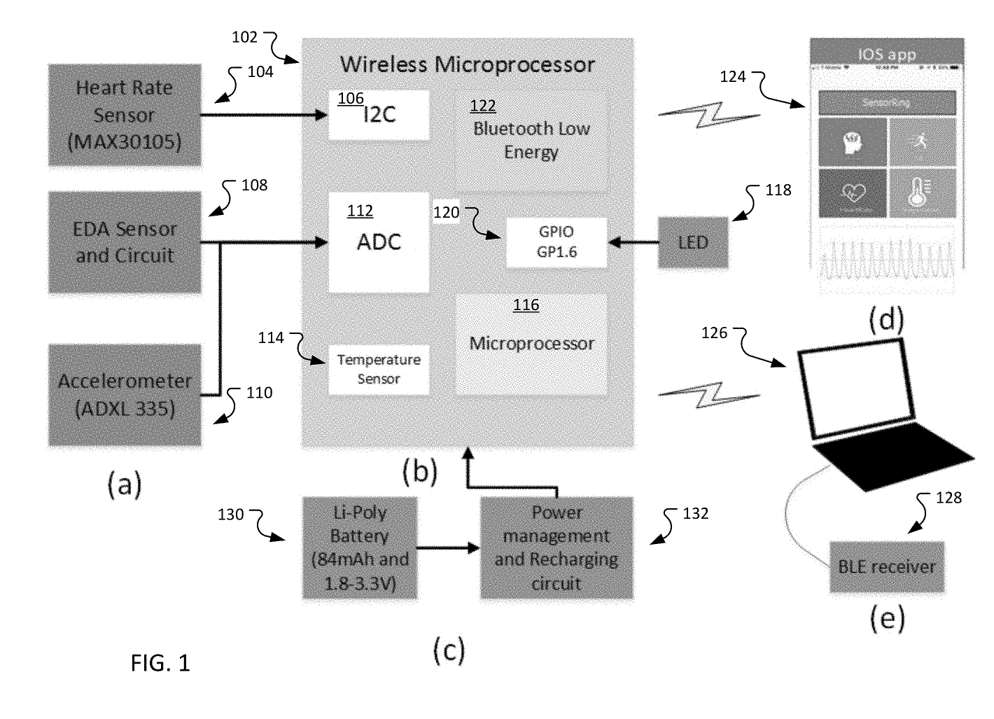

FIG. 1 is a block diagram of the sensor architecture for Wearable biosensor ring. The ring contains a wireless microprocessor 102. Connected to the wireless microprocessor 102 is a heart rate sensor 104. An example of a suitable heart rate sensor is, for example, MAX30105 from Maxim Integrated Inc. The wireless microprocessor 102 may communicate with the heart rate sensor 104 using the I2C standard 106. The ring also contains an T and circuit 108 and an accelerometer 110. The EDA Sensor and Circuit 108 and the Accelerometer 110 may communicate with the wireless microprocessor using ADC 112. The wireless microprocessor may include a temperature sensor 114 and a microprocessor 116.

The wireless microprocessor 102 may communicate with LED devices 118 using GPIO, GP1.6 120. The wireless microprocessor may also include the ability to communicate using Bluetooth Low Energy 122. The wireless microprocessor 102 may communicate with, for example, an application 124 running on a smart device (such as a phone, watch, etc.). The wireless microprocessor 102 may also communicate with a computer system 126 that has a blue tooth low energy receiver 128. The wireless microprocessor 102 may get power from a LI-Poly Battery 130 integrated with a power management and recharging circuit 132.

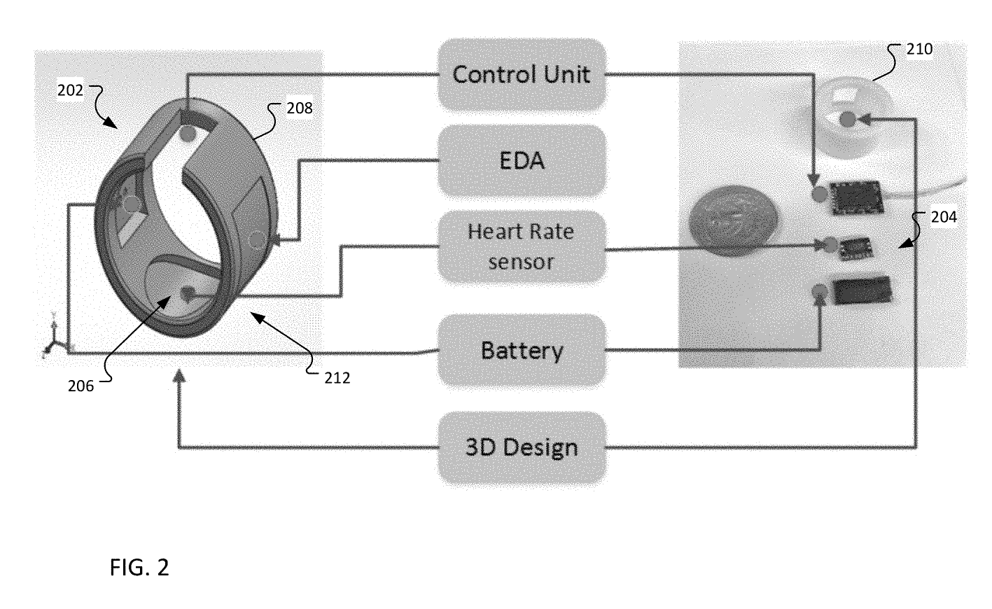

FIG. 2 illustrates a 3D model 202 of the wearable biosensor ring. Different ring sizes have been designed for testing. The ring has been designed to fit all the electronic components without making it bulky and irritating to the wearer's skin while providing accurate results. In some implementations, the outer radius of Wearable biosensor ring can be between 16.5-19.5 mm and the inner radius can be 15-18 mm, with a constant thickness of 1.5 mm and width of 17 mm. The printing was carried out with commercial available 3D printer with Acylonitrile Butadiene Styrene (ABS) thermoplastic materials. ABS objects are a strong oil-based plastic material and widely used for 3D printing and toy manufacturing.

In some implementations, the heart rate sensor 204 can integrated into the wearable ring sensor (such as, for example, MAX30105 from Maxim Integrated Inc). The heart rate sensor 204 may be enclosed in body 208, 210 of the ring, for example at location 206. It can also includes a pulse oximeter. Due to its extremely small surface mounted size (for example, 6×3×2 mm) and its ability to communicate through a I2C standard, it was well suited for the proposed system. The sensor can include internal red, green and infrared LED's, photodiodes, optical electronics and better ambient light rejection. The Wearable biosensor ring can pulse different LEDs and capturing the reflected signal. Based on the reflected light, the Wearable biosensor ring can detect blood volume change (i.e. heart rate and oxygen level).

In some implementations, the ring may include a button 212. The button may be used to send a help signal to a computer or other device, using, for example, low energy Bluetooth signals. The receiving computer may then contact a predetermined person, including, for example, a predetermined contact person or emergency services. In some implementations, the accelerometer within the ring may detect when a person has fallen, for example, based on sudden jarring motions. The ring may be programmed to request help, as described above, unless the wearer takes an action within a predetermined period of time (for example, standing up).

EDA is correlated to a person's stress level and it has been used on many other occasions in polygraph tests for drug use detection. Sweat is electronically conductive and an increase in sweat quantity can be referred to as a decrease in skin resistance or an increase in skin conductance. This resistance can be measured with two metal contacts placed on the skin. It is better to use electrodes that are non-reactive to skin therefore, in this study, we used a flexible silver material. The electrodes should be placed where the human body has the highest density of eccrine sweat such as the forehead, armpits, feet or fingers. Therefore, the proposed ring sensor can be one of the best candidates for high accuracy EDA measurement.

Note that the physical movement of the electrodes may cause motion artifacts, which will lead to inaccurate results. Burring and filtering of the analog can be performed before the signal was digitized through the microprocessor. The skin conductance can easily be converted to voltage signal using a voltage divider circuit. The skin resistance usually varies from 50 Kohm to 10 Mohm. Consequently, the buffers should match or have a higher impedance than the human skin. Due to the high input impedance, additional buffering and amplifications were applied. For example, The MC33501 (Onsemi, RI, USA) can be used to buffer the signal. It is small in size (2.9×1.3×0.9 mm), has low leakage current and an extremely low power op-amp (can operate only on 1 V). The two leads for EDA measurement can be placed inside the ring, and a voltage divider can be used to convert resistance to voltage. The signal can then passes through a band-pass filter of 0.45 to 4.5 Hz to auto-tune and remove high frequency noise coming from the source. The signal can be processed by an ADC of microprocessor to digitalize and sent through Bluetooth. The readings were averaged to smooth out the values.

The accelerometer can gather information by measuring acceleration in the three axes with a built-in local coordinate system. In some implementations, ADXL335 (Analog Devices) has been used as a tri-axes accelerometer to get motion information. The accelerometer is low-power, small in size (4 mm×4 mm×1.45 mm) and easy to use. It is connected to the microprocessor on three analog pins, and capable of measuring the full range of ±3 g. The temperature was also monitored in this work. Its sensor is integrated with a wireless microprocessor.

The wireless microprocessor can be a high performance smart Bluetooth Low Energy (BLE) radio transceiver and capable of communicating with a mobile device or PC browser. The main CPU can be a 32-bit ARM Cortes M0 processor, that has a 10-bit internal ADC. The purpose of using this module is due to its miniaturized size (10 mm×7 mm), low-power, data security and great scalability. In some implementations, the Wearable biosensor ring does not contain any USB connectivity, however to program and debug the microprocessor, an over the air (OTA) solution can be used. Operating voltage for the wireless module is 1.8V-3.3V and it has high resolution and low latency up to 30 meters. Its relatively small size and inexpensive characteristics make it easy to develop a prototype for the embedded system. To connect the wireless microprocessor to a computer, it can use Gazell or GZLL protocol, which was developed by Nordic Semiconductor.

Software Architecture

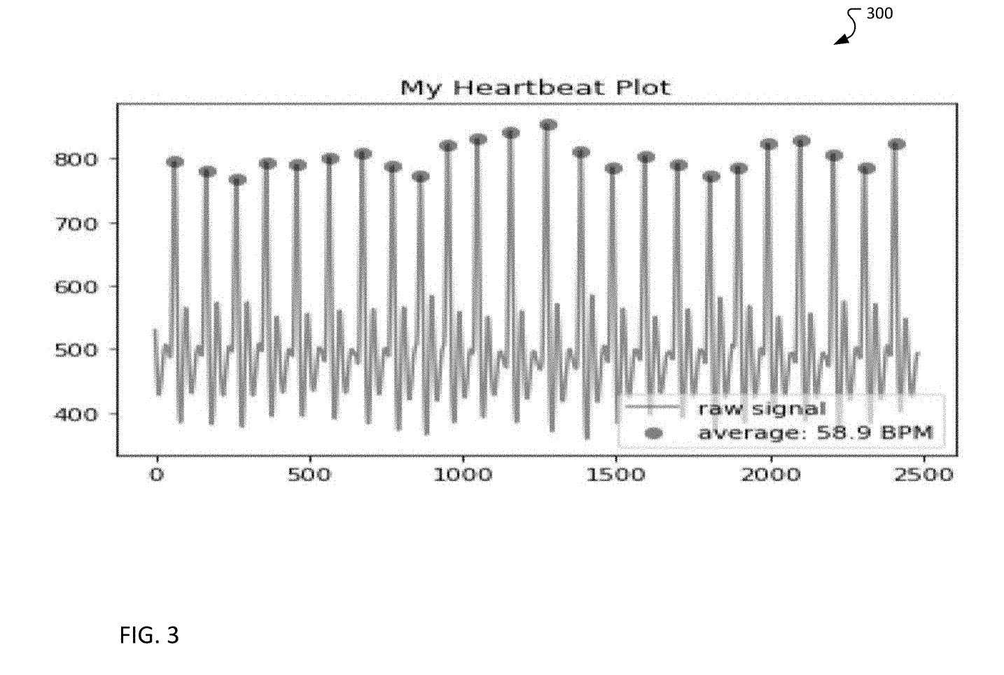

In modern days, health care consumers can record and use a range of data through wearable devices, smart textiles, and intelligent sensors that can be worn on the body such as a watch or wristband. The most popular clinical cardiac signal analysis is electrocardiogram (ECG). ECG is an electrical measurement which provides a rich screening tool for a wide range of cardiac abnormalities due to its detailed information about a particular group of cardiac muscle activity. However, it is very complex and requires multiple points of physical contact to acquire a clean and robust signal, especially when there is an extreme size constraint that is a fundamental requirement of the wearable biosensor. On the other hand, optical sensors, like Photoplethysmogram (PPG), measure arterial volume with a single contact point. This enables an easy continuous measurement of heart rate for wearable biosensors. Therefore, PPG can be a potential solution for the need of a readily available tool for heart rate measurement. The PPG working principle is emitting light through the skin and subcutaneous tissue with a specific wavelength, such as an LED. The light going through blood volume, either will be absorbed or reflected in the source. The photodiode will determine how much light has been absorbed and reflected back, and transduce the measured light intensity of an electrical signal. With each cycle of cardiovascular system, the blood volume changes. It causes the expansion and contraction of blood vessels resulting in a PPG wave form as shown in FIG. 3. As this waveform 300 is continuous and periodic with each cardiac cycle, it can be used to calculate heart rate.

The heart rate signals can be obtained from Wearable biosensor ring, x where is n is the size of the detection window. In some implementations, n=15 seconds with 75% of overlapping window. The sampling rate for the collection was 250 Hz, and about 3750 samples were collected from the PPG sensor. The signal x passes through an IIR filter to detect an accurate rhythm, and it also maximizes peak components and reduces noise. The IIR filter is designed to be a fifth order band-pass Butterworth filter with a bandwidth of 1 to 20 Hz to extract a clear heartbeat (HR(x[n]). HR(x[n]) is applied to the differentiation model to measure the slope and maximum of the peak of the incoming signal, called dHR.

dHR=HR(x[n+1])−Hr(x[n]) (1)

The output signal (dHR[n]) goes through a nonlinear transformation (sHR[n]), which boosts the main peaks of the desired signal and utilizes a dynamic threshold system. Lastly, a moving average technique is applied to detect the peaks. In this implementation, the moving average is suitable because the R-peaks can change due several events such as when the wearer is in motion. At the same time, PPG has also had a secondary peak that might give a false positive and can be detected as a R-peak. Therefore, the system can ignore any consecutive peaks if they are too close to the first peak. The system can also optimize the moving average so that it best fits in this particular application.

Electrodermal activity (EDA) is simply a response of the human body to outer stimuli that result in continuous changes in skin resistance. EDA is also known as galvanic skin resistance (GSR) it can be used as a biomarker for emotional changes that result in the sudden change in skin resistance. The EDA is linked to the automatic nervous system (ANS) which, for example, higher stimulation of ANS will cause a quick fall in EDA and lower stimulation will show the opposite. In depth, the reason behind the changes skin conductance is due to secretion of specific type of sweat gland that is related to the ANS by the human body. Such as when people go through different phases of emotional state (i.e. Stressed, anxiety, embarrassed and happiness) their EDA changes drastically, making it a primary biomarker to measure ANS activity.

After hardware level filtration and buffering, the system can use a baseline wandering correction. The reason for the use of the correction can be related to sweat secretion over a long period of time. To minimize the baseline shifting and make the experiment more accurate for an individual wearer, we subtract the mean from EDA. After the baseline correction, we measure the peaks of EDA by taking the derivative of the peaks. Any value less than the threshold has been nullified. Then the changes in points for rising slope or falling slope are counted as a detected peak. The SenosRing sends the processed data to the host via Bluetooth low energy. However, these are still raw values that do not have any unit. EDA is measured in microSiemens (μS), but before we calculate EDA, first we have to extract skin conductance (in Ohm) from equation 2 and 3.

$$\begin{matrix}SR ( Ohm ) = 1 - \frac{ADC}{{2}^{n}} & ( 2 ) \\ EDA ( μ S ) - \frac{1}{SR} & ( 3 )\end{matrix}$$

Where, SR=sensor resistance in ohms

EDA=EDA value in micro-Siemens

ADC=analog value measured from Wearable biosensor ring

n=Number of bits in the channel

Along with EDA and heart rate, the Wearable biosensor ring can also captures the three-axis acceleration and the skin temperature of the wearer. Because motion and thermal information are capable of influencing the sweat glands as well as a cardiovascular activity, they can provide more insights into ANS activity.

### IV. RESULTS

A study was done that was permitted by the Institutional review board (IRB) at the University of Massachusetts Dartmouth. All of the volunteers were selected from the Bioengineering, Electrical and Computer Engineering departments in Univ. of Massachusetts Dartmouth. All of the participants were 18 and older and were provided the full right to keep or destroy the collected data of them using Wearable biosensor ring. Volunteers were given a brief description of the Wearable biosensor ring, explanation of the purpose of the study, and their informed written consent agreement was collected. The sample contains 42 participants, which includes 23 men and 20 women, ages between 19 and 26 years. The volunteers about 165-176 lbs with height ranging from 168-176 cms. 67% of them were unemployed and 33% were employed. The survey also included the data of the last stressful event each participant experienced, indications of illicit drug use and the timing of the exams. The survey was collected to measure the emotional conditions of the wearer as well as to understand data for future research. We excluded any volunteers who had a record of drug abuse, mental illness, cardiovascular diseases and was feeling uncomfortable to wear the Wearable biosensor ring.

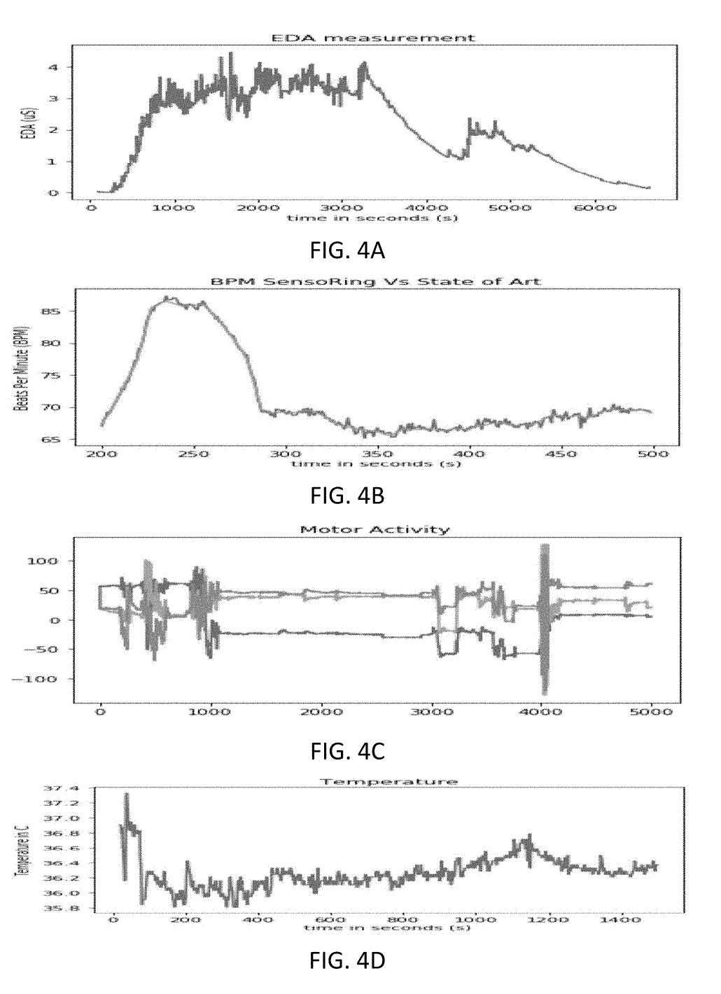

The output of the proposed system is shown in FIGS. 4A-D. The overall quality of the signal was good, and all of the data were processed using Python notebook. Then a GUI was developed to capture and analyze the data in real-time. The raw data coming from the system were re-sampled and filtered to reduce motion artifacts and electrical noise. 86% of the participants reported that they prefer a ring sensor instead of a watch due its comfortness, high accuracy and wearability. An example of typical EDA signal with Wearable biosensor ring is shown in FIG. 4A. EDA also changes drastically during exercise, especially during the cycling task experiment that was followed by an increase in skin conductance level (SCL). Changes in skin conductance responses (SCR) were also noticed during the experiment. To validate the performance of the system, we also correlated the measured data from Wearable biosensor ring with BITalino. BITalino is commercially available development board specialized in monitoring vital signs, including EDA, ECG and acceleration. The measured EDA results had a high correlation coefficient (0.94<r<0.99) during the experiment. Within the range of human skin conductance, the mean sensitivity of the Wearable biosensor ring is 0.01+0.01 μS, which is comparable to the reference device (0.01 μS).

The graph in FIG. 4B compares the beats per minute (BPM) or heart rate (HR) values obtained by both BITalino and the proposed system over a period of 5 minutes. In this work, the deviation of the BPM values from the two devices never crossed 3 BPM. The table below shows the results in time domain for BITalino and Wearable biosensor ring after processing the signal. The results are relatively compatible for both the mean of the RR and the standard deviation.

TABLE
Comparison of time series data from
Wearable biosensor ring and BITalino.

| | Mean RR (ms) | | | | STD RR (ms) | | | |
|---|---|---|---|---|---|---|---|---|
| # | Ring | Ref. | Err. | Acc. % | Ring | Ref | Err. | Acc. % |
| 1 | 708.5 | 721.4 | 1.8 | 98.2 | 132.4 | 133.6 | 0.88 | 99.1 |
| 2 | 705.2 | 719.3 | 1.9 | 98 | 92.35 | 93.6 | 1.39 | 98.6 |
| 3 | 736.1 | 740.6 | 0.6 | 99.4 | 94.65 | 97.2 | 2.66 | 97.3 |
| 4 | 758.6 | 768.5 | 1.3 | 98.7 | 146.2 | 148.5 | 1.48 | 98.5 |
| 5 | 726.5 | 744.8 | 2.5 | 97.5 | 106.2 | 107.1 | 0.75 | 99.3 |
| . . . | . . . | . . . | . . . | . . . | . . . | . . . | . . . | . . . |
| 40 | 741.5 | 764.5 | 3 | 97 | 109.3 | 111.2 | 1.71 | 98.3 |
| 41 | 726.9 | 745.2 | 2.5 | 97.5 | 88.4 | 89.45 | 1.22 | 98.8 |
| 42 | 733.7 | 760.6 | 3.5 | 96.5 | 99.7 | 99.85 | 0.19 | 99.8 |
| 43 | 729.6 | 745.3 | 2.1 | 97.9 | 115.9 | 117.5 | 1.33 | 98.7 |
| Av. | 775.4 | 789.5 | 1.8 | 98.2 | 109.4 | 110.8 | 1.3 | 98.7 |

Average of mean RR intervals for BITalino and Wearable biosensor ring are 789.5 and 775.4, respectively. Also, average for the standard deviation of RR intervals is 110.89 for BITalino and 109.47 for Wearable biosensor ring. The accuracy for both mean RR and STD RR are comparatively high (more than 90%). The locomotion increases sharply during exercise and warming up session, as shown in FIG. 4C. Motor activity can provide information about activity and the gestures of the user. During the experiment there were no significant changes in skin temperature (FIG. 4D). However, that particular result can be due to sweat. In general, the BITalino shows better results, but results from Wearable biosensor ring are very similar and with further testing along with additional prototypes, errors could be eradicated over time.

The major portions of these unavoidable noises come from motion artifacts. Hence, one of the important factors in this research is to classify the output signals. Similar work has been done before, but most of the solutions are non-real-time or do not support IoT. However, it is impossible to compare with other researches due to differences in experimental setup.

### V. CONCLUSION

The wearable sensor ring is a compact and low-cost wearable ring that can measure EDA, HR, locomotion and temperature with a single node. The wearable sensor ring enables the use of a finger as a recording place for certain biosignals, the miniaturization of the full system, as well as the design of the proposed system that allows for unobtrusive assessment of physiological biomarkers. Experimental results from the proposed system were highly correlated with the reference module. The wearable sensor ring can integrate with computer applications (for example, an application can execute on assorted smart phone and other computing devices.). The applications can allow others (such as a physician) to monitor the wearer from a remote location. This can unlock long-term monitoring of emotional states of patients using wearable sensors. A recent study shows that EDA and temperature play a vital role in detecting illicit drug intake. In the next phase of our research, we will be studying on illicit drug detection using Wearable biosensor ring. Moreover; there is the possibility that we commercialize this device in the upcoming year as an unobtrusive health monitoring and diagnosing system for adults and infants. The proposed system is IoT enabled, which makes it a strong candidate for remote health monitoring.

What is claimed is:

1. A ring comprising:
a body,
a microprocessor, located within the body;
an EDA sensor in communication with the microprocessor and located within the body.

2. The ring of claim 2, wherein the body further includes a pulse oximeter in communication with the microprocessor.

3. The ring of claim 1, wherein the thickness of the body is less than 2 mm.

4. The ring of claim 1, further comprising a button, wherein the microprocessor is configured to send a signal to a computing system in response to the button being pressed.

5. The ring of claim 4, wherein the signal is used to alert a third party.

6. The ring of claim 1, further comprising an accelerometer.

7. The ring of claim 6, wherein the microprocessor is configured to identify signals of the accelerometer consistent with the wearer falling.

8. The ring of claim 1, wherein the ring is in communication with a computer system, the computer system configured to maintain health history information provided by the ring.

9. The ring of claim 1, wherein the microprocessor is configured to monitor the physiological condition of the wearer.

10. A ring comprising:
a body,
a microprocessor, located within the body;
a pulse oximeter in communication with the microprocessor and located within the body.

11. The ring of claim 10, wherein the thickness of the body is less than 2 mm.

12. The ring of claim 10, further comprising a button, wherein the microprocessor is configured to send a signal to a computing system in response to the button being pressed.

13. The ring of claim 12, wherein the signal is used to alert a third party.

14. The ring of claim 10, further comprising an accelerometer.

15. The ring of claim 12, wherein the microprocessor is configured to identify signals of the accelerometer consistent with the wearer falling.

16. The ring of claim 10, wherein the ring is in communication with a computer system, the computer system configured to maintain health history information provided by the ring.

17. The ring of claim 10, wherein the microprocessor is configured to monitor the physiological condition of the wearer.
